(19) United States
(12) Patent Application Publication
(10) Pub. No.: US 2020/0352124 A1
(43) Pub. Date: Nov. 12, 2020

(54) METHOD OF PRODUCING PATHOGEN-FREE CANNABIS PLANTS AND PATHOGEN-FREE PLANTS AND CLONES PRODUCED THEREFROM

(71) Applicants: Daniel J. Grace, San Leandro, CA (US); William J. Roberts, Oakland, CA (US)

(72) Inventors: Daniel J. Grace, San Leandro, CA (US); William J. Roberts, Oakland, CA (US)

(21) Appl. No.: 16/685,630

(22) Filed: Nov. 15, 2019

Related U.S. Application Data
(63) Continuation-in-part of application No. 16/013,787, filed on Jun. 20, 2018, now abandoned.

(51) Int. Cl. A01H 4/00

(52) U.S. Cl. CPC A01H 4/005; A01H 5/02; A01H 4/008

(57) ABSTRACT

Disclosed herein are methods of producing substantially pathogen-free plants of the genus Cannabis and pathogen-free plants and clones produced therefrom. One embodiment of the method comprises pretreating a progenitor plant of the genus Cannabis, surface sterilizing a shoot segment of the pretreated plant with a bleach solution, excising a meristematic tip of the shoot segment, and transferring the meristematic tip into a culturing plate comprising a supplemented Murashige and Skoog culture medium for further culturing. The supplemented Murashige and Skoog culture medium can comprise benzyladenine, naphthaleneacetic acid, gibberellic acid, or a combination thereof.

### CROSS REFERENCE TO RELATED APPLICATION

This application is a continuation in part of U.S. application Ser. No. 16/013,787, filed Jun. 20, 2018, which is incorporated by reference herein in its entirety.

### TECHNICAL FIELD

This disclosure relates generally to the field of plant husbandry and cultivation, more specifically, to methods of producing substantially pathogen-free plants of the genus Cannabis and substantially pathogen-free plants and clones produced from such methods.

### BACKGROUND

The legalization of Cannabis for recreational and medicinal use in states such as Colorado, Washington, Oregon, Alaska, and California has resulted in a deluge of growers entering the Cannabis cultivation space. However, the rise in Cannabis plant stock as a whole has also brought along an attendant rise in pathogen infection rates among such plant populations. While viruses such as Tobacco Mosaic Virus (TMV), Cannabis Cryptic Virus (CCV), and Hop Mosaic Virus (HpMV) have been known to infect plants of the genus Cannabis, a recently discovered pathogen has begun to raise alarms in numerous grow-houses, nurseries, and farms. Those in the Cannabis industry refer to the new pathogen as Putative Cannabis Infectious Agent or PCIA and plants infected with PCIA are often referred to as “duds.” Applicants are credited for the recent discovery that the new pathogen is Hop latent viroid (HpLVd); accordingly, previously-named PCIA will herein be referred to as HpLVd.

HpLVd is a viroid first reported infecting Hops (Humulus lupulus) in 1988 (Puchta et al 1988). Its effects on Hops can vary from no detectable symptoms to a catastrophic decline in plant growth and secondary metabolite production (Pethybridge et al 2008). Until recently the only species confirmed to be hosts of HpLVd are two species in the Humulus genus and stinging nettle, though Matouek (2001, 2003) was able to experimentally transmit heat-mutated versions of HpLVd onto tomatoes. Viroids in general are considered one of the most rapidly evolving biological systems on Earth and thus are often able to infect multiple and diverse host plants (Diener 1995).

Unsurprisingly almost all published research on HpLVd concerns its impact on Hops. Research on HpLVD transmission in Hops has found it to be most easily transmitted through propagation of infected plant material and mechanically via cultivation equipment with other potential vectors such as leaf contact and insects having very low to no transmissibility (Pethybridge et al 2008, Barbara and Adams 2003). Testing of samples from commercial cultivation operations has since shown HpLVd to be widely distributed in Cannabis, perhaps even globally. With the discovery of HpLVd in Cannabis, and the associated negative economic impacts, a method of eradicating it is needed to rid commercially important cultivars of HpLVd.

FIG. 1 is a black-and-white image of a side-by-side comparison of a healthy Cannabis sativa plant on the right side and a Cannabis sativa plant infected by HpLVd on the left. As shown in FIG. 1, the infected plant exhibits a lack of apical dominance (i.e., the side branches grow more than the central stem), diminished leaf size, bowed branches or excessive branching, increased internode spacing, and brittle stems. Infected plants also often do not produce flowers that mature and do not produce the type of resins or oils typical of healthy Cannabis plants.

Plant epidemiological studies conducted of farms in the Humboldt, Calif. region in 2015 revealed that up to 20% to 35% of plants in the farms surveyed were infected with HpLVd. HpLVd has been known to spread by plant-on-plant contact, insect vectors, and human or tool contact with infected plants. The ease and speed by which HpLVd can spread in both contained and outdoor plant environments have made the pathogen a prime concern for the Cannabis industry.

While HpLVd has been attributed to one or more viruses or viroids, studies are still ongoing to understand the pathogen. The uncertainty concerning HpLVd has opened the door for many unproven treatment methods to take hold within the Cannabis industry. Such treatment methods include immersing diseased plants or plant parts in alcohol, continuously replacing the soil of diseased plants, and the excessive use of harmful insecticides. However, such methods have not been shown to reliably reduce the incidence of HpLVd or viral infections in treated plant populations. Moreover, such methods may worsen the problem when diseased plants treated by such ineffective methods are introduced back into the nursery stock.

Therefore, improved methods of producing substantially pathogen-free plants of the genus Cannabis are needed. In addition, such methods should be cost-effective and easy to implement on a large-scale. Moreover, such a method should result in plant populations that exhibit, in general, a lower rate of pathogen infections and are robust and healthy.

### SUMMARY

Methods of producing substantially pathogen-free plants of the genus Cannabis and substantially pathogen-free plants and clones produced from such methods are disclosed herein.

One embodiment of the invention relates to a method of producing plants of the genus Cannabis, the method can include: pretreating a progenitor plant of the genus Cannabis, resulting in a pretreated plant; surface sterilizing a shoot segment of the pretreated plant with a bleach solution; excising a meristematic tip of the shoot segment; and transferring the meristematic tip into a culturing plate comprising at least one plant hormone capable of inducing formation of a whole plant from the meristematic tip.

In some embodiments, the pretreating step can include one or more of heating the progenitor plant within a heating chamber, cooling the progenitor plant within a cooling chamber, delivering an electrical current to the progenitor plant, and/or applying an anti-viral compound, such as Ribavirin, to the progenitor plant. The pretreatment step can include more than one treatments concurrently or in sequence. The treatments can be separated by meristem excision and regrowth.

One embodiment of the method can comprise pretreating a progenitor plant of the genus Cannabis within a heating chamber resulting in a heat-treated plant. The progenitor plant can be in a vegetative growth stage when heated. Heating the progenitor plant can comprise heating the progenitor plant at alternating temperatures of approximately 100° F. and 85° F. The progenitor plant can be heated at each temperature for approximately a four-hour period for a total of 14 days. The progenitor plant can be between approximately 6 inches and 18 inches in height, as measured from the soil surface, when subjected to heat-treatment.

The method can further comprise surface sterilizing a shoot segment of the heat-treated plant with a bleach solution. Surface sterilizing the shoot segment of the heat-treated plant can comprise immersing the shoot segment in the bleach solution for between approximately 10 minutes and 20 minutes or, more specifically, 15 minutes. The bleach solution can comprise approximately 2.475% (w/v %) of sodium hypochlorite.

The method can also comprise excising a meristematic tip of the shoot segment. Excising the meristematic tip of the shoot segment can comprise excising an apical portion of the shoot segment equal to or less than approximately 0.5 mm in size. The apical portion of the shoot segment can comprise meristem tissue. The meristematic tip can be excised using a scalpel under microscopy. The method can further comprise transferring the meristematic tip into a culturing plate comprising a supplemented suitable plant growth medium (e.g., Murashige and Skoog culture medium) for further culturing. The supplemented\culture medium can comprise benzyladenine (6-benzylaminopurine), naphthaleneacetic acid, and gibberellic acid. In one embodiment, the supplemented culture medium can comprise 1.0 mg/L of benzyladenine (6-benzylaminopurine), 0.1 mg/L of naphthaleneacetic acid, and 0.1 mg/L of gibberellic acid.

The method can further comprise transferring a plantlet grown from the meristematic tip from the culturing plate into a test tube comprising additional supplemented plant growth medium after approximately 21 days to 30 days. The method can also comprise transferring the plantlet growing in the test tube from the test tube into a large-tissue culture vessel comprising plant growth medium after approximately 28 days to 56 days.

The method can further comprise transferring the plantlet growing in the large-tissue culture vessel into a first rooting medium after 28 days to 56 days to yield a young elite mother plant. In addition, the method can comprise transferring the young elite mother plant and at least a portion of the first rooting medium into a second rooting medium after approximately 10 days to 16 days and growing the young elite mother plant in the second rooting medium between 7 days and 28 days to yield an elite mother plant.

A pathogen-free, surface-sterile, regenerated plant of the genus Cannabis is also disclosed. The plant is produced by a process comprising the steps of pretreating a progenitor plant of the genus Cannabis.

The process can further comprise surface sterilizing a shoot segment of the heat-treated plant with a bleach solution. Surface sterilizing the shoot segment of the heat-treated plant can comprise immersing the shoot segment in the bleach solution for between approximately 10 minutes and 20 minutes or, more specifically, 15 minutes. The bleach solution can comprise approximately 2.475% (w/v %) of sodium hypochlorite.

The process can also comprise excising a meristematic tip of the shoot segment. Excising the meristematic tip of the shoot segment can comprise excising an apical portion of the shoot segment equal to or less than approximately 0.5 mm in size. The apical portion of the shoot segment can comprise meristem tissue. The meristematic tip can be excised using a scalpel under microscopy.

The process can further comprise transferring the meristematic tip into a culturing plate comprising a supplemented Murashige and Skoog culture medium for further culturing. The supplemented Murashige and Skoog culture medium can comprise benzyladenine (6-benzylaminopurine), naphthaleneacetic acid, and gibberellic acid. In one embodiment, the supplemented Murashige and Skoog culture medium can comprise 1.0 mg/L of benzyladenine (6-benzylaminopurine), 0.1 mg/L of naphthaleneacetic acid, and 0.1 mg/L of gibberellic acid.

The process can further comprise transferring a plantlet grown from the meristematic tip from the culturing plate into a test tube comprising additional supplemented Murashige and Skoog culture medium after approximately 21 days to 30 days. The process can also comprise transferring the plantlet from the test tube into a large-tissue culture vessel comprising Murashige and Skoog culture medium after approximately 28 days to 56 days.

The process can further comprise transferring the plantlet growing in the large-tissue culture vessel into a first rooting medium after 28 days to 56 days to yield a young elite mother plant. In addition, the process can comprise transferring the young elite mother plant and at least a portion of the first rooting medium into a second rooting medium after 10 days to 16 days and growing the young elite mother plant in the second rooting medium between 7 days and 28 days to yield an elite mother plant.

A cloned plant of the genus Cannabis is also disclosed. The cloned plant is produced by a process comprising the steps of pretreating a progenitor plant of the genus Cannabis.

The process can further comprise surface sterilizing a shoot segment of the heat-treated progenitor plant with a bleach solution. Surface sterilizing the shoot segment of the heat-treated plant can comprise immersing the shoot segment in the bleach solution for between approximately 10 minutes and 20 minutes or, more specifically, 15 minutes. The bleach solution can comprise approximately 2.475% (w/v %) of sodium hypochlorite.

The process can also comprise excising a meristematic tip of the shoot segment. Excising the meristematic tip of the shoot segment can comprise excising an apical portion of the shoot segment equal to or less than approximately 0.5 mm in size. The apical portion of the shoot segment can comprise meristem tissue. The meristematic tip can be excised using a scalpel under microscopy.

The process can further comprise transferring the meristematic tip into a culturing plate comprising a supplemented plant growth medium (e.g., Murashige and Skoog culture medium) for further culturing. The supplemented medium can comprise benzyladenine (6-benzylaminopurine), naphthaleneacetic acid, and gibberellic acid. In one embodiment, the supplemented medium can comprise 1.0 mg/L of benzyladenine (6-benzylaminopurine), 0.1 mg/L of naphthaleneacetic acid, and 0.1 mg/L of gibberellic acid.

The process can further comprise transferring a plantlet grown from the meristematic tip from the culturing plate into a test tube comprising additional supplemented medium after approximately 21 days to 30 days. The process can also comprise transferring the plantlet from the test tube into a large-tissue culture vessel comprising medium after approximately 28 days to 56 days.

The process can further comprise transferring the plantlet growing in the large-tissue culture vessel into a first rooting medium after 28 days to 56 days to yield a young elite mother plant. In addition, the process can comprise transferring the young elite mother plant and at least a portion of the first rooting medium into a second rooting medium after 10 days to 16 days and growing the young elite mother plant in the second rooting medium between 7 days and 28 days to yield an elite mother plant.

The process can also comprise obtaining a stem cutting of the elite mother plant and immersing at least a segment of the stem cutting in a rooting hormone solution. The segment of the stem cutting can be immersed in the rooting hormone solution for between approximately 5 seconds and 10 seconds. In some embodiments, the rooting hormone solution can comprise indole-3-butyric acid and 1-napthaleneacetic acid as active ingredients.

The process can further comprise transferring the stem cutting into a temperature-controlled rooting medium and further cultivating the stem cutting in the temperature-controlled rooting medium until roots form to yield the cloned plant. The temperature-controlled rooting medium can be a rock-wool rooting medium and the temperature of the rock-wool rooting medium can be heated and maintained at approximately 80° F. In some embodiments, the rock-wool rooting medium can be heated by a heating system (e.g., a hydronic heating system) or heating mat.

### BRIEF DESCRIPTION OF THE DRAWINGS

FIG. 1 is a black-and-white image of a side-by-side comparison of a healthy plant of the genus Cannabis on the right and a plant infected by HpLVd on the left.

FIG. 2A illustrates certain steps of an embodiment of a method for producing substantially pathogen-free plants of the genus Cannabis.

FIG. 2B illustrates certain other steps of the embodiment of the method for producing substantially pathogen-free plants of the genus Cannabis.

FIG. 3A is a black-and-white image of an embodiment of a temperature-controlled growth chamber heating one or more progenitor plants.

FIG. 3B illustrates an embodiment of an example heating schedule undertaken by the temperature-controlled growth chamber to heat the one or more progenitor plants.

FIG. 4 is a black-and-white image of heat-treated plants.

FIG. 5A is a black-and-white image of a shoot segment of a heat-treated plant.

FIG. 5B illustrates an embodiment of a technique of surface sterilizing the shoot segment.

FIG. 6 illustrates the anatomy of a distal portion of a shoot segment comprising a meristematic tip.

FIG. 7A is a black-and-white image of plantlets grown from meristematic tips on culturing plates comprising supplemented Murashige and Skoog culture medium.

FIG. 7B is a black-and white image of a plantlet growing within a test tube comprising supplemented Murashige and Skoog culture medium.

FIG. 8A is a black-and-white image of three types of culturing vessels used as part of the method of producing substantially pathogen-free plants including culturing plates, test tubes, and large tissue-culture vessels.

FIG. 8B is a black-and-white image of young elite mother plants grown from meristematic tips and acclimated for ambient ex-vitro growing conditions.

FIG. 9A is a black-and-white image of a miniature-sized elite mother plant for further propagation via cloning.

FIG. 9B is a black-and-white image of a regular-sized elite mother plant for further propagation via cloning.

FIG. 10 illustrates an embodiment of certain steps of a method for producing cloned plants from substantially pathogen-free elite mother plants.

FIG. 11A is a black-and-white image of a stem cutting obtained from an elite mother plant.

FIG. 11B is a black-and-white image of a segment of the stem cutting immersed in a rooting hormone solution.

FIG. 12A is a black-and-white image of the stem cutting in a rooting medium.

FIG. 12B is a black-and-white image showing the stem cutting being further cultivated in a temperature-controlled rooting medium to yield a cloned plant.

FIG. 13 is a table showing the results of tests conducted on plants of the genus Cannabis produced by the methods described herein for incidence of HpLVd infections.

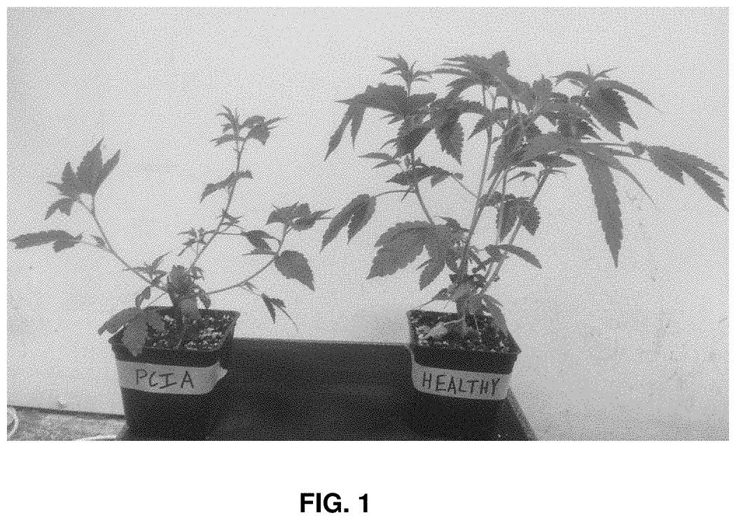

### DETAILED DESCRIPTION

Methods of producing substantially pathogen-free plants of the genus Cannabis and substantially pathogen-free plants and clones produced from such methods are disclosed herein. In some embodiments, the method can include a pretreatment step selected from the group consisting of heat treatment, cold treatment, electricity treatment, anti-viral compound treatment or combinations thereof. In some embodiments, treatments are used in sequence or simultaneously. In some embodiments, treatments are separated by a meristem excision step. For example, the plant is pretreated with one treatment and the meristem is excised. Once the meristem has grown out (to a length of 1-4 cm), the plant is pretreated with a second treatment. In some embodiments, more than one pretreatment step is used. In some embodiments, no pretreatment is used.

One embodiment of the method can include pretreating a progenitor plant of the genus Cannabis.

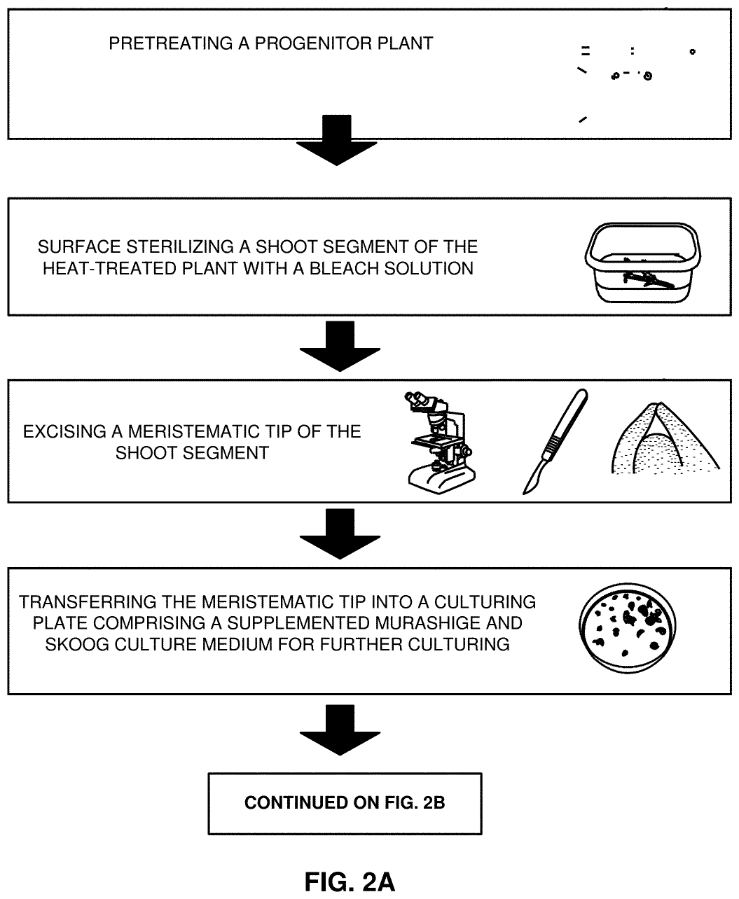

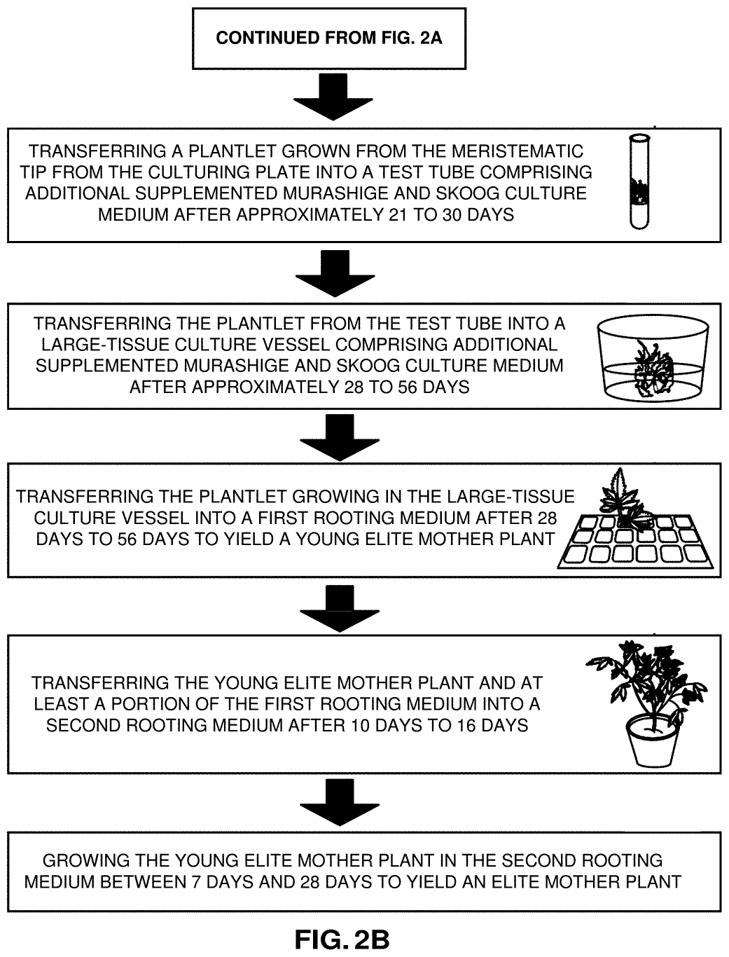

FIG. 2A illustrates certain steps of an embodiment of a method for producing substantially pathogen-free plants of the genus Cannabis. Embodiments of the methods described herein can be applied to Cannabis sativa plants, Cannabis indica plants, or hybrids thereof.

### Heat Treatment

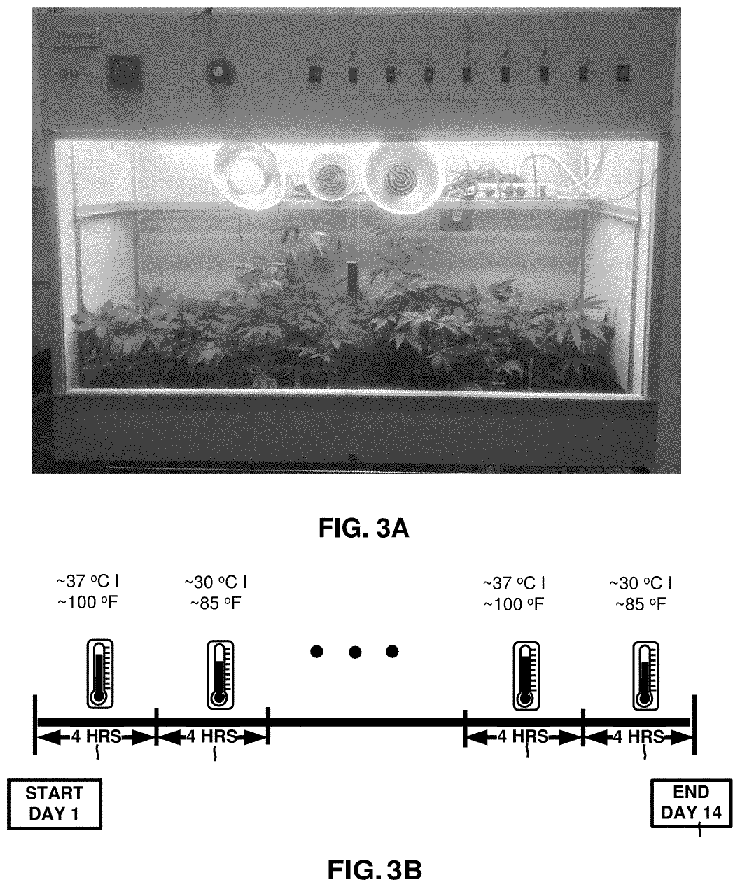

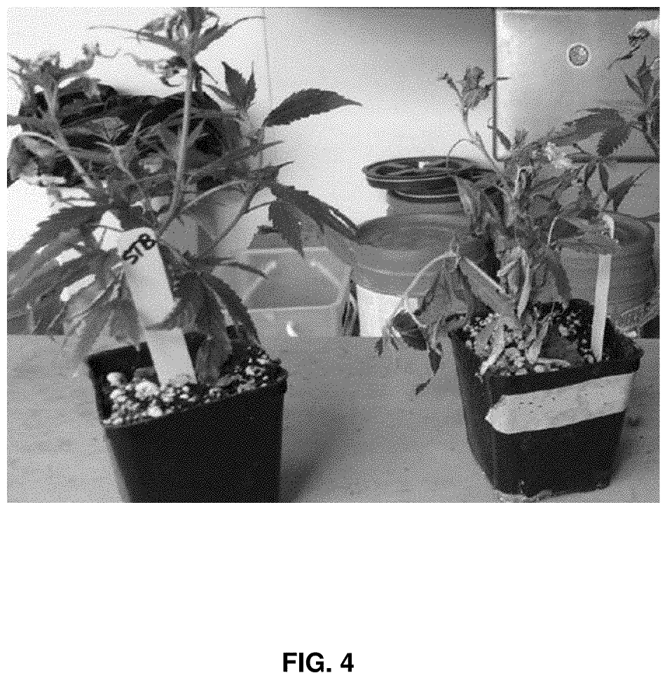

In some embodiments, the method can include pretreating a progenitor plant with heat. The method can comprise heating a progenitor plant (see FIG. 3A) of the genus Cannabis within a heating chamber (see FIG. 3A) resulting in a heat-treated plant (see FIG. 4). The progenitor plant can be in a vegetative growth stage when pretreated. In certain embodiments, the progenitor plant can be between the ages of 2 weeks to 3 weeks when pretreated. In other embodiments, the progenitor plant can be between the ages of 3 weeks to 4 weeks when pretreated. The progenitor plant can be heated according to a heating schedule (see FIG. 3B). The heating schedule and the heating chamber will be discussed in more detail in following sections.

### Cold Treatment

In some embodiments, the method can include pretreating a progenitor plant with cold temperatures within a cold chamber. The progenitor plant can be in a vegetative growth stage when cooled. The cold chamber can be a refrigerator. The cold chamber can include a light source. The light source can be LED or fluorescent, and/or the like. The light source can prevent the plant from entering into flowering. The plant can be grown in in vitro, for example in plastic or glass test tubes. The plant can be grown with or without roots. The plant can be 1-10 cm in length. The progenitor plant can be cooled according to a cooling schedule. The cooling schedule can be 24 hours a day for 1, 2, 3, 4, 5, 6, 7, 8, 9, 10, 11, 12 or more months. The chamber can be kept at a temperature of 5-10 C.

### Electrotherapy

In some embodiments, the method can include pretreating a progenitor plant with electrotherapy in a chamber capable of delivering current to the plant. The progenitor plant can be in a vegetative growth stage when treated. The chamber can be a gel electrophoresis tank. The chamber can include a solution capable of carrying an electrical current. The solution can be a salt such as sodium chloride or potassium chloride, or the like. The solution can have a concentration of 0.5M-2M. The current can be applied for any combination of 5-30 min at currents of 25-100 mA milliamperes (mA). Current can be supplied from a DC electrophoresis power supply capable of delivering precise levels of electrical current. The plant must be of a size capable of immersion in the chamber. In some embodiments, the plant can be cut into stem segments of 1-2 cm with at least one growth node. There can be as many as three growth nodes. The stem can be immersed into the solution while the current is applied. Immediately after the treatment the meristems are excised.

In some embodiments, no chamber or solution is used. The electrical current is delivered to the plant by direct contact of component capable of carrying the current such as a metal clip.

Heat Treatment Combined with Electrotherapy

In some embodiments, a progenitor plant is pretreated with both heat in a heating chamber as described above and electrotherapy in a chamber capable of delivering current to the plant as described above. Treatments can occur in sequence or simultaneously. In some embodiments, the plant is pretreated with heat treatment as described above, then immediately treated with electricity as described above. After the treatments, the meristem is excised, as described above.

Cold Treatment Combined with Electrotherapy

In some embodiments, a progenitor plant is pretreated with both cold in a cooling chamber as described above and electrotherapy in a chamber capable of delivering current to the plant as described above. Treatments can occur in sequence or simultaneously. Treatments can be separated by a meristem excision step. In some embodiments, the plant is pretreated with electrotherapy, followed by meristem excision. Once the meristem has grown out, the plant is pretreated with cold treatment.

Heat Treatment Followed by, Preceding, or Alternating with Cold Treatment

In some embodiments, a progenitor plant is pretreated with both heat and cold in either order or alternating between the two treatments. In some embodiments, the plant is treated with heat ex-vitro, followed by meristem excision, then cold treatment, then another meristem excision.

### Combination Treatment Combining Heat Treatment, Cold Treatment, and Electrotherapy

In some embodiments, a progenitor plant is pretreated with heat and cold in either order or alternating between the two treatments, further combined with electrotherapy either in sequence or simultaneously with heat or cold or both. In some embodiments, the plant is treated with heat, then treated with electrotherapy, followed by meristem excision. Once the plants have grown out, they are treated with cold treatment and another meristem excision. This protocol considers the way the plants go in and out of sterile tissue culture.

Heat Treatment Combined with Use of Anti-Viral Compound(s) and Optional Electrotherapy.

In some embodiments, a progenitor plant is pretreated with both heat in a heating chamber and an anti-viral compound. The anti-viral compound can be Ribavirin or any other suitable anti-viral compound. In some embodiments, 10-75 mg/L of an anti-viral compound is added to the culture medium to regenerate the excised meristems after heat treatment. Electrotherapy can optionally also be combined with these other treatments.

Cold Treatment Combined with Use of Anti-Viral Compound(s) and Optional Electrotherapy.

In some embodiments, a progenitor plant is pretreated with both cold in a cooling chamber and an anti-viral compound. The anti-viral compound can be Ribavirin or any other suitable anti-viral compound. In some embodiments 10-75 mg/L anti-viral compound is included in the culture medium in the test tubes containing the plant which undergoes cold treatment. Electrotherapy can optionally also be combined with these other treatments.

Electrotherapy Combined with Anti-Viral Compounds

In some embodiments, a progenitor plant is pretreated with both electrotherapy in a chamber capable of delivering current to the plant and an anti-viral compound. In some embodiments, 10-75 mg/L of the antiviral compound is added to the culture medium used to regenerate the excised meristems post-electrotherapy treatment.

### Combination Treatment Combining Heat Treatment, Cold Treatment, Electrotherapy, and Anti-Viral Compounds

In some embodiments, a progenitor plant is pretreated with heat and in either order or alternating between the two treatments, further combined with electrotherapy either in sequence or simultaneously with heat or cold or both. In some embodiments, the plant is treated with heat, followed by electrotherapy, followed by meristem excision and cultured in media containing 10-75 mg/L anti-viral compound. Once the plants have grown out, they are then placed in cold treatment of 1-12 mounts in media with or without the anti-viral compound. Once the treatment is completed, the meristems are again excised and cultured on media with or without anti-viral compound.

In some embodiments, the progenitor plant can be a plant infected by a pathogen. In one embodiment, the pathogen can be HpLVd. In other embodiments, the progenitor plant can be a plant infected by another pathogen such as a virus from the family Virgaviridae, a virus from the family Betaflexiviridae, a viroid from the family Pospiviroidae, a viroid from the family Avsunviroidae, a phytoplasma, and/or the like, and/or a combination thereof. For example, the progenitor plant can be a plant infected by a pathogen such as a virus from the genus Tobamovirus, a virus from the genus Carlavirus, a viroid from any of the genera Pospiviroid, Hostuviroid, Cocadviroid, Apscaviroid, or Coleviroid, a parasitic bacteria from the genus Candidatus Phytoplasma, or a combination thereof. In these and other embodiments, the progenitor plant infected by the one or more pathogens can be exhibiting symptoms of infection or disease or be in an asymptomatic stage or phase. In further embodiments, the progenitor plant can be a healthy plant of the genus Cannabis having been cultivated near or in proximity to other plants infected by the pathogen.

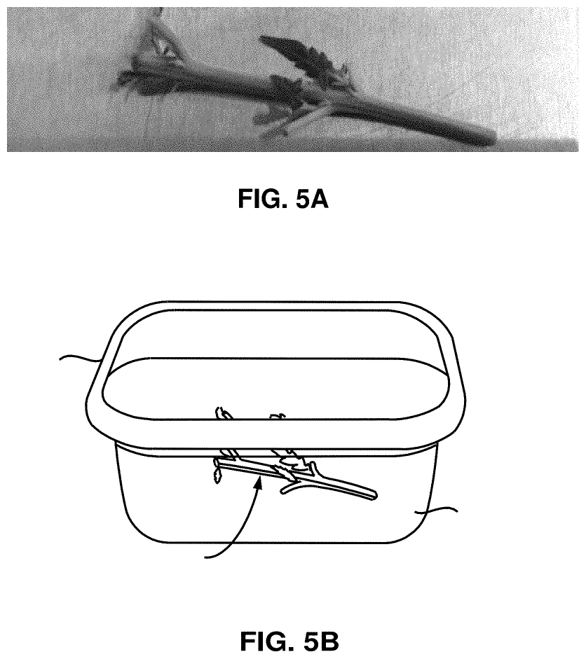

The method can also comprise surface sterilizing a shoot segment (see FIG. 5A) of the pre-treated plant with a bleach solution (see FIG. 5B). Surface sterilizing the shoot segment with the bleach solution will be discussed in more detail in the following sections.

The method can further comprise excising a meristematic tip (see FIG. 6) of the sterilized instance of the shoot segment. The meristematic tip can be excised using a scalpel under microscopy. Excising the meristematic tip will be discussed in more detail in the following sections.

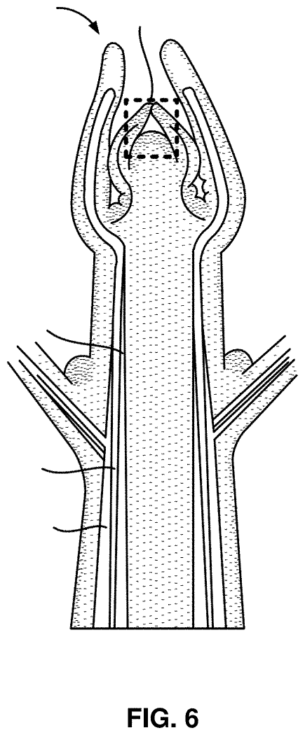

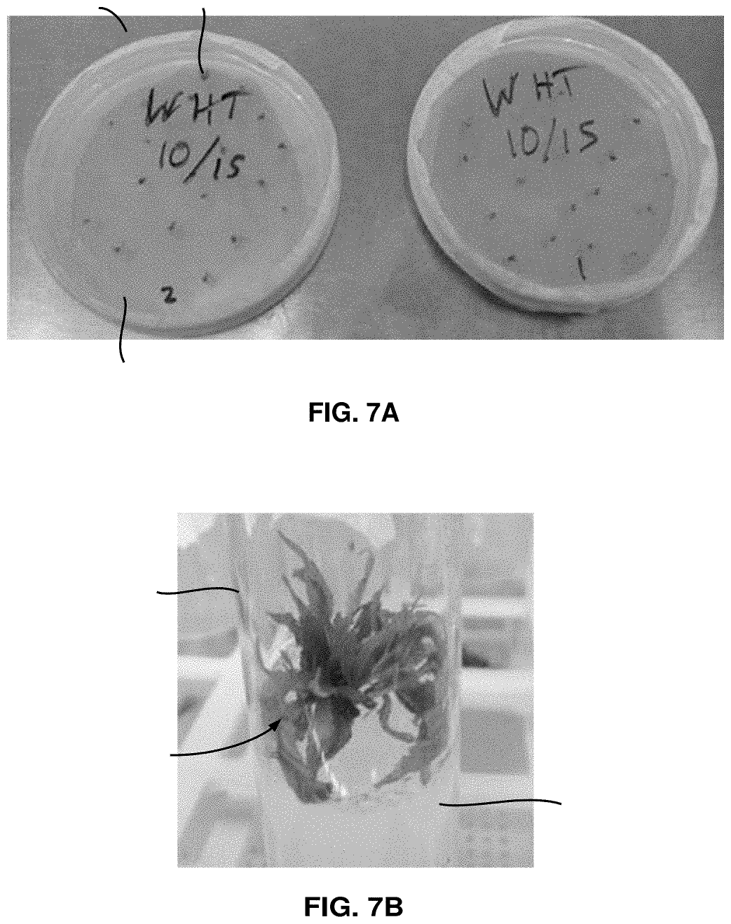

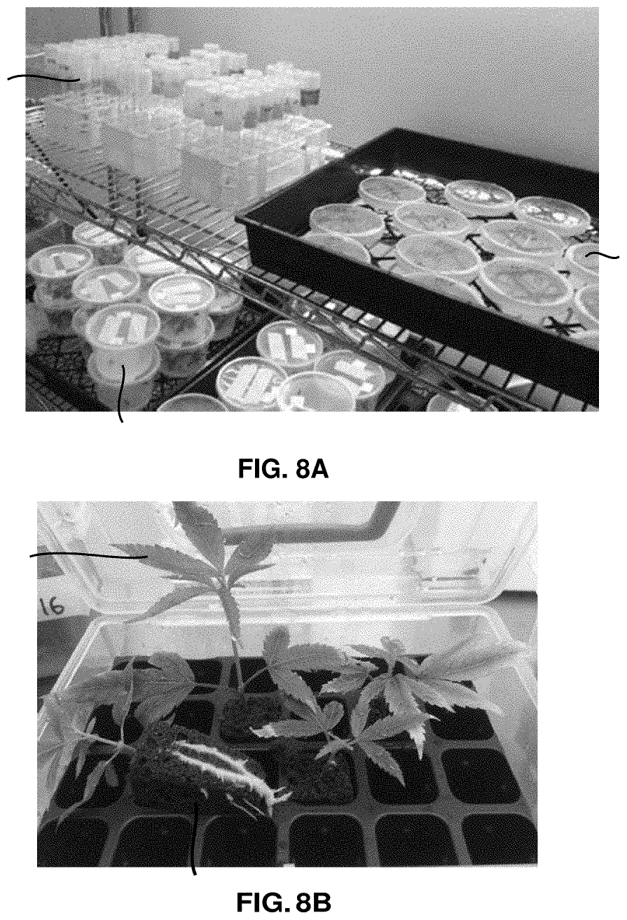

The method can also comprise transferring the meristematic tip into a culturing plate (see FIGS. 7A and 8A) comprising a supplemented plant growth medium (e.g, Murashige and Skoog culture medium) for further culturing. Any suitable plant grown media can be used. The supplemented Murashige and Skoog culture medium can comprise growth regulators configured to support the growth and development of nascent plant cells and coordinate intercellular communication. The supplemented Murashige and Skoog culture medium will be discussed in more detail in the following sections.

FIG. 2B illustrates certain additional steps of the embodiment of the method for producing substantially pathogen-free plants of the genus Cannabis. The method can further comprise transferring a plantlet grown from the meristematic tip from the culturing plate into a test tube comprising additional supplemented medium after approximately 21 days to 30 days (or 3-4 weeks). The method can also comprise transferring the plantlet from the test tube into a large-tissue culture vessel (see FIG. 8A) comprising culture medium after approximately 28 days to 56 days (or 4-8 weeks).

In addition, the method can further comprise transferring the plantlet growing in the large-tissue culture vessel (see FIG. 8A) into a first rooting medium (see FIG. 8B) after 28 days to 56 days to yield a young elite mother plant in step 214. As will be discussed in more detail in the following sections, an “elite mother plant” can be a substantially pathogen-free mother plant produced from the methods described herein that can be used to create cloned plants or genetic copies (e.g., through cuttings). A “young elite mother plant” can be an immature or developing elite mother plant that is not yet ready to produce cuttings.

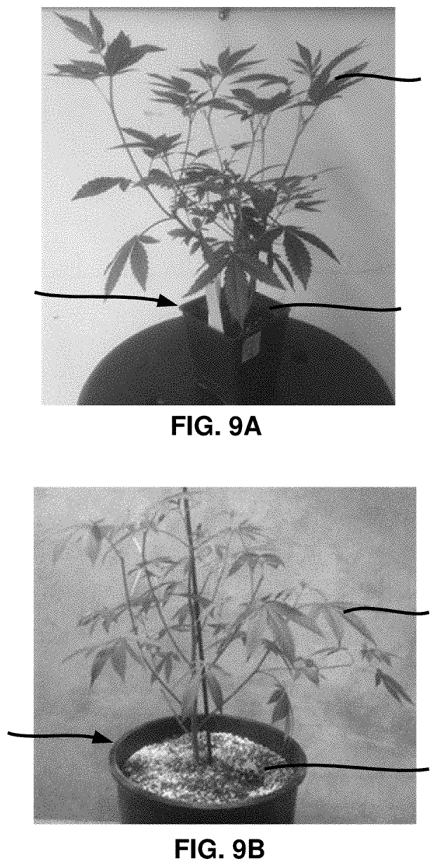

The method can also comprise transferring the young elite mother plant and at least a portion of the first rooting medium into a second rooting medium (see FIGS. 9A and 9B) after approximately 10 days to 16 days in the first rooting medium. For example, the first rooting medium can be a rock-wool cube and at least a portion of the rock-wool cube (or the entire rock-wool cube) can be transplanted into the second rooting medium. In some embodiments, the second rooting medium can comprise soil, pumice, perlite, peat, coir, polymer stabilized rooting plugs, other types of mineral wool, or any combination thereof.

The method can further comprise growing the young elite mother plant in the second rooting medium between approximately 7 days and 28 days to yield an elite mother plant (see FIGS. 9A and 9B). As will be discussed in more detail in the following sections, the elite mother plant can be used to produce cuttings to produce cloned plants or genetic copies.

FIG. 3A is a black-and-white image of an embodiment of a heating chamber used to heat the one or more progenitor plants. In some embodiments, the heating chamber can be a temperature-controlled growth chamber designed for heating plant matter. For example, the heating chamber can be a ThermoFisher Scientific™ Growth Chamber (Model No. 3768 or Catalog No. 846). In other embodiments, the heating chamber can be any heating chamber having a temperature control thermostat and programmable heating element.

In some embodiments, the heating chamber can have sliding glass doors for ease of access and viewing. Moreover, the heating chamber can undertake a 24-hour heating cycle such that the heating element is constantly on during the heat treatment period.

In certain embodiments, the interior of the heating chamber can comprise a number of fluorescent or incandescent lamps with adjustable lighting levels. Moreover, the heating chamber can also comprise a number of fans to circulate air within the interior of the heating chamber.

In one embodiment, the heating element can be a heating strip positioned within an interior of the heating chamber. In other embodiments, the heating element can comprise heat lamps, heating fans, or a combination thereof.

In some embodiments, the progenitor plants can be heated at a relative humidity of approximately 10-70%. In other embodiments, the progenitor plants can be heated at a relative humidity of between approximately 10% and 60%. In additional embodiments, the progenitor plants can be heated at a relative humidity of between approximately 10% and 50%. In alternative embodiments, the progenitor plants can be heated at a relative humidity of between approximately 20% and 50%.

As previously discussed, the progenitor plants can be plants infected by a pathogen. In one embodiment, the pathogen can be HpLVd. In other embodiments, the progenitor plant can be a plant infected by another pathogen such as a virus from the family Virgaviridae, a virus from the family Betaflexiviridae, a viroid from the family Pospiviroidae, a viroid from the family Avsunviroidae, a phytoplasma, or a combination thereof. For example, the progenitor plant can be a plant infected by a pathogen such as a virus from the genus Tobamovirus, a virus from the genus Carlavirus, a viroid from any of the genera Pospiviroid, Hostuviroid, Cocadviroid, Apscaviroid, or Coleviroid, a parasitic bacteria from the genus Candidatus Phytoplasma, or the like, or a combination thereof.

In these and other embodiments, the progenitor plant infected by the one or more pathogens can be exhibiting symptoms of infection or disease or be in an asymptomatic stage or phase. Given that certain Cannabis plants may be infected by HpLVd other pathogens and exhibit little or no symptoms during the vegetative growth stage, the method can also involve heating healthy plants that are suspected of being infected by HpLVd or other pathogens. Such plants can be those cultivated near or in proximity to other symptomatic plants infected by HpLVd or other pathogens.

FIG. 3B illustrates an embodiment of a heating schedule undertaken by the heating chamber to heat the one or more progenitor plants. The heating schedule can comprise heating the one or more progenitor plants at a first heating temperature for a first heating duration, adjusting the heating temperature to a second heating temperature and heating the one or more progenitor plants at the second heating temperature for a second heating duration, and repeating this alternating heating cycle until a total heating period has elapsed.

In some embodiments, the first heating temperature can be between approximately 95° F. (35° C.) and 104° F. (40° C.). For example, the first heating temperature can be approximately 100° F. (37.78° C.). In these and other embodiments, the second heating temperature can be between approximately 80° F. (26.67° C.) and 88° F. (31.11° C.). For example, the second heating temperature 310 can be approximately 85° F. (29.44° C.).

In some embodiments, the first heating duration can be between approximately 3.75 hours to 4.25 hours. For example, the first heating duration can be approximately 4 hours. In these and other embodiments, the second heating duration can be approximately 3.75 hours to 4.25 hours. For example, the second heating duration can be approximately 4 hours. In certain embodiments, the first heating duration can be the same as the second heating duration. In alternative embodiments, the first heating duration can differ from the second heating duration. As a more specific example, the heating schedule can comprise heating the one or more progenitor plants at alternating temperatures of approximately 100° F. and 85° F. where the progenitor plants are heated at each temperature for approximately a four hour period for a total of 14 days.

In alternative embodiments not shown in FIG. 3B, the heating schedule can comprise heating the one or more progenitor plants at a constant heating temperature of between approximately 95° F. (35° C.) and 104° F. (40° C.). For example, the heating schedule can comprise heating the one or more progenitor plants at a constant heating temperature of approximately 100° F. (37.78° C.) for the total heating period.

In some embodiments, the total heating period can be between approximately 13 days (approximately 312 hours) and 15 days (360 hours). For example, the total heating period can be approximately 14 days (approximately 336 hours).

One unexpected discovery is that numerous cultivars of Cannabis sativa, Cannabis indica, and hybrids thereof can be heat treated using the heating schedule disclosed herein without substantially impairing the viability of the meristematic tips of such heat-treated plants for further culturing. Moreover, the heating schedule disclosed herein has been discovered to be optimal for the method of producing substantially pathogen-free plants of the genus Cannabis or clones thereof.

FIG. 4 is a black-and-white image of embodiments of heat-treated plants. As shown in FIG. 4, the heated-treated plants can be in a stressed state after undergoing heat treatment. One objective of subjecting the progenitor plants to the heat treatment disclosed herein is to slow the progress of any infectious agents or pathogens which may have infected the progenitor plants. More specifically, one objective of subjecting the progenitor plants to the heat treatment disclosed herein is to prevent the infectious agent or pathogen or byproducts produced thereby from reaching the shoot meristem of the heated progenitor plants.

As previously disclosed, the progenitor plants can be in a vegetative growth stage when heated. For example, the progenitor plant can be between the ages of 2 weeks to 3 weeks when heated. In other example embodiments, the progenitor plant can be between the ages of 3 weeks to 4 weeks when heated. Such plants can have a height dimension of between approximately 6 inches (approximately 15.24 cm) and 18 inches (approximately 45.72 cm), as measured from the soil surface. One unexpected discovery made by the applicant is that progenitor plants of the height dimension (between approximately 6 inches (approximately 15.24 cm) and 18 inches (approximately 45.72 cm)) disclosed herein are mostly able to withstand the heating schedule 304 disclosed herein without suffering irreparable harm to their meristematic tissue. Thus, Cannabis progenitor plants of the height dimension disclosed herein are optimal for the heating step of the method disclosed herein for producing substantially pathogen-free plants of the genus Cannabis or clones thereof.

FIG. 5A is a black-and-white image of a shoot segment excised from the heat-treated plant. The shoot segment can be a segment of a main stem of the heat-treated plant. The shoot segment can also be a segment of a side shoot or branch of the plant. The shoot segment can measure between approximately 3.0 inches (7.62 cm) and 5.0 inches (12.7 cm) in length. The shoot segment can be excised using sterilized scalpels, shears, knives, or a combination thereof.

FIG. 5B illustrates an embodiment of a technique of surface sterilizing the excised shoot segment. In the embodiment shown in FIG. 5B, surface sterilizing the excised shoot segment can comprise immersing the excised shoot segment in a bleach solution. For example, a container can be filled with the bleach solution and the excised shoot segment can be completely immersed in the bleach solution. In other embodiments, at least part of the excised shoot segment such as the top of the shoot segment is immersed in the bleach solution. The excised shoot segment can be immersed in the bleach solution for between approximately 10 minutes and 20 minutes. In one embodiment, the excised shoot segment can be immersed in the bleach solution for approximately 15 minutes. In alternative embodiments, the excised shoot segment can be sprayed with the bleach solution or the bleach solution can be poured on the excised shoot segment.

The bleach solution can comprise approximately between 0.825% and 2.475% (w/v %) of sodium hypochlorite. The bleach solution can be made by diluting a bleach solution (e.g., HDX™ Germicidal Bleach or Clorox™ Germicidal Bleach) comprising approximately 8.25% (w/v %) of sodium hypochlorite (which yield 7.86% of available chlorine) with distilled water. For example, the bleach solution can be made by combining approximately 30.0% (v/v %) germicidal bleach solution comprising 8.25% sodium hypochlorite with approximately 70.0% (v/v %) distilled water. The end result is a bleach solution comprising approximately 2.475% (w/v %) of sodium hypochlorite. In another example, a 10% (v/v %) germicidal bleach solution can be made by combining approximately 8.25% sodium hypochlorite with approximately 90.0% (v/v %) distilled water. The end result is a bleach solution comprising approximately 0.825% (w/v %) of sodium hypochlorite.

FIG. 6 illustrates the anatomy of a distal portion of a shoot segment of a plant of the genus Cannabis. FIG. 6 illustrates a location of the meristematic tip relative to the rest of the distal portion of the shoot segment. The method can comprise excising the meristematic tip using a sterilized scalpel under microscopy. The meristematic tip can refer to an apical portion of the shoot segment equal to approximately 0.50 mm in length. In other embodiments, the meristematic tip can refer to an apical portion of the shoot segment less than approximately 0.50 mm in length such as between 0.30 mm and 0.50 mm. In alternative embodiments, the meristematic tip can refer to an apical portion of the shoot segment greater than 0.50 mm in length such as between approximately 0.55 mm and 0.75 mm.

In some embodiments, the meristematic tip can comprise the apical dome and a limited number of young leaf primordia. The meristematic tip excludes any differentiated provascular tissues or vascular tissues. For example, care should be taken not to excise any part of the shoot segment comprising the procambium, xylem, or phloem.

The objective of excising the meristematic tip is to excise meristem tissue without excising any part of the vasculature of the plant that may comprise viruses, viroids, or other pathogens. One advantage of pre-treating the plant prior to excision is that the pretreatment slows the progress of any viral or other pathogen infections and ideally prevents the pathogen from reaching the shoot apical meristem of the plant.

The method can comprise transferring the excised meristematic tips (also referred to as the meristem explants) into culturing plates (see FIG. 7A) comprising supplemented Murashige and Skoog culture medium. When initially placed on the culturing plates, the meristematic tips can be barely perceptible to the naked eye.

FIG. 7A is a black-and-white image of meristematic tips that have been growing on culturing plates comprising supplemented Murashige and Skoog culture medium for approximately two weeks. In some embodiments, the culturing plates can be transparent Petri dishes such as borosilicate or polyethylene Petri dishes. In these and other embodiments, the culturing plates can be a substantially circular Petri dish having an outer diameter of between approximately 2.75 inches (approximately 70.0 mm) and 3.93 inches (approximately 100 mm) and a plate depth of approximately 0.6 inches (approximately 15 mm). In alternative embodiments, the culturing plates can be compartmentalized or be substantially rectangular in shape.

As shown in FIG. 7A, multiple meristematic tips can be cultured within the same culturing plate. Moreover, as shown in FIG. 7A, the culturing plates can be covered by plastic wrap, cling wrap, or a combination thereof to prevent contamination and moisture loss.

The supplemented plant growth medium can be any medium suitable for plant growth. For example, the medium can be supplemented Murashige and Skoog culture medium which can comprise Murashige and Skoog culture medium supplemented with plant growth-regulators. Murashige and Skoog culture medium is plant culture medium having ingredients similar to those presented in Murashige, Toshio, and Folke Skoog. “A revised medium for rapid growth and bio assays with tobacco tissue cultures.” Physiologia Plantarum 15.3 (1962): 473-497. Several other culture media formulations can also be used, such as Gamborg's Culture Medium (Gamborg 1976) and White's Basal Medium (White 1943). Commercially available culture media formulations can include those distributed by ThermoFisher Scientific Inc., Sigma-Aldrich Co. LLC, or W.W. Grainger, Inc.

The supplemented Murashige and Skoog culture medium can comprise plant growth-regulators including benzyladenine (6-benzylaminopurine), naphthaleneacetic acid, gibberellic acid, or a combination thereof. In one embodiment, the supplemented Murashige and Skoog culture medium can comprise plant growth-regulators in the amount of 1.0 mg/L of benzyladenine (6-benzylaminopurine), 0.1 mg/L of naphthaleneacetic acid (1-naphthaleneacetic acid), and 0.1 mg/L of gibberellic acid. The growth regulators can be configured to support the growth and development of nascent plant cells and coordinate intercellular communication.

FIG. 7B is a black-and white image of a plantlet grown from a meristematic tip within a test tube comprising supplemented Murashige and Skoog culture medium. The method can comprise transferring a plantlet grown from the meristematic tip from the culturing plate 700 into the test tube after approximately 21 to 30 days.

In some embodiments, the test tube can be a plastic test tube made in part of polystyrene, polyethylene, or a combination thereof. In other embodiments, the test tube can be a glass test tube made in part of boro silicate glass (e.g., Pyrex™ test tubes). In some embodiments, the test tube can have a tube diameter of between approximately 0.98 inches (25.0 mm) and 1.50 inches (38.0 mm). The test tube can be filled partially with supplemented Murashige and Skoog culture medium but have plenty of room for the plantlet to grow within the test tube.

FIG. 8A is a black-and-white image of three types of culturing vessels used as part of the method of producing substantially pathogen-free plants including culturing plates, test tubes, and large tissue-culture vessels. The method can further comprise transferring the plantlet from a test tube into a large-tissue culture vessel comprising Murashige and Skoog culture medium after approximately 28 days to 56 days.

In some embodiments, the large tissue-culture vessels can be substantially cylindrical in shape. In other embodiments, the large tissue-culture vessels can be substantially frustoconic in shape. For example, the large tissue-culture vessels can have a carrying capacity of between approximately 350 mL and 500 mL. When the large tissue-culture vessel is substantially shaped as an upside-down frustoconic, the tissue-culture vessel can have a top diameter of approximately 4.625 inches (approximately 11.75 cm) and a bottom diameter of approximately 3.375 inches (approximately 8.57 cm).

In some embodiments, the large tissue-culture vessels can be made in part from polyethylene, polypropylene, or a combination thereof. In other embodiments, the large tissue-culture vessels can be made in part from borosilicate glass.

FIG. 8B is a black-and-white image of young elite mother plants grown from cultured meristematic tips excised from heat-treated plants and acclimated for ambient ex-vitro growing conditions. As shown in FIG. 8B, the young elite mother plants are rooted in a first rooting medium. The young elite mother plants are developed from the plantlets grown in the large tissue-culture vessels shown in FIG. 8A.

As previously discussed, the plantlets can be transferred from the large-tissue culture vessels (see FIG. 8A) into the first rooting medium as part of the method. The can be transferred from the large-tissue culture vessels (see FIG. 8A) into the first rooting medium after approximately 28 days to 56 days spent in the large-tissue culture vessels. Once the plantlets have taken root in the first rooting medium, the plantlets can be considered young elite mother plants.

In some embodiments, the first rooting medium can be rock-wool (e.g., a rock-wool cube) or other types of mineral wool. In these and other embodiments, the first rooting medium can also comprise soil, pumice, perlite, peat, coir, polymer stabilized rooting plugs, or any combination thereof.

For purposes of this disclosure, an “elite mother plant” can be a substantially pathogen-free mother plant of the genus Cannabis produced from the methods and treatment steps described herein. Elite mother plants can be used to create cloned plants or genetic copies through cuttings and other propagation methods. A “young elite mother plant” can be an immature or developing elite mother plant that is not yet ready for cloning or propagation.

The method can also comprise transferring the young elite mother plant and at least a portion of the first rooting medium into a second rooting medium (see FIGS. 9A and 9B) after approximately 10 days to 16 days in the first rooting medium. For example, the first rooting medium can be a rock-wool cube and at least a portion of the rock-wool cube (or the entire rock-wool cube) can be transplanted along with the young elite mother plant into the second rooting medium. In one embodiment, the second rooting medium can be soil. In this and other embodiments, the second rooting medium can also comprise pumice, perlite, peat, coir, polymer stabilized rooting plugs, other types of mineral wool, or any combination thereof.

The method can further comprise growing the young elite mother plants in the second rooting medium to yield an elite mother plant (see FIGS. 9A and 9B). The young elite mother plants can be allowed to grow in the second rooting medium for between approximately 7 days and 28 days. As will be discussed in more detail in the following sections, the elite mother plant can be harvested for cuttings to produce cloned plants or genetic copies.

One unexpected discovery is that elite mother plants produced, in part, by the method disclosed herein yielded, on a population level, a greater percentage of phenotypically healthier propagates or clones than mother plants produced by other methods. Another unexpected discovery is that elite mother plants produced, in part, by the method disclosed herein produced, on average, more cannabinoid or terpenoid-rich plant matter than mother plants produced by other methods.

FIG. 9A is a black-and-white image of a miniature-sized elite mother plant (also referred to as a mini-elite mother plant). A miniature-sized elite mother plant can be an elite mother plant grown in a small container. For example, the small container can be an upside-down truncated square pyramidal container having a top length and top width dimension of approximately 3.5 inches (or approximately 8.89 cm), a height dimension of approximately 3.375 inches (or approximately 8.57 cm), and a bottom length and width dimension of approximately 2.5 inches (or approximately 6.35 cm). In other embodiments, the small container can be a cylindrical or frustoconic container having a top diameter of approximately 3.5 inches (or approximately 8.89 cm). In additional embodiments, the miniature-sized elite mother plants 900 can be grown in a substantially cuboidal container having a length, height, and width dimension of between approximately 3.0 inches (or approximately 7.62 cm) and 5.0 inches (or 12.7 cm).

From the time the young elite mother plants are transplanted into the small containers 904, the young elite mother plants can be allowed to grow in the small containers 904 for between approximately 7 days and 21 days (or 1 week to 3 weeks) until the branches of such plants are long enough to be harvested for cuttings.

FIG. 9B is a black-and-white image of a regular-sized elite mother plant. A regular-sized elite mother plant can be an elite mother plant grown in a large container. For example, the regular-sized elite mother plant can be grown in a container or pot having a container volume of between approximately 5 gallons (approximately 18.9 liters) and 7 gallons (approximately 26.5 liters). From the time the young elite mother plants are transplanted into the large containers, the young elite mother plants can be allowed to grow in the large containers for between approximately 14 days and 28 days (or 2 weeks to 4 weeks) until the branches of such plants are long enough to be harvested for cuttings.

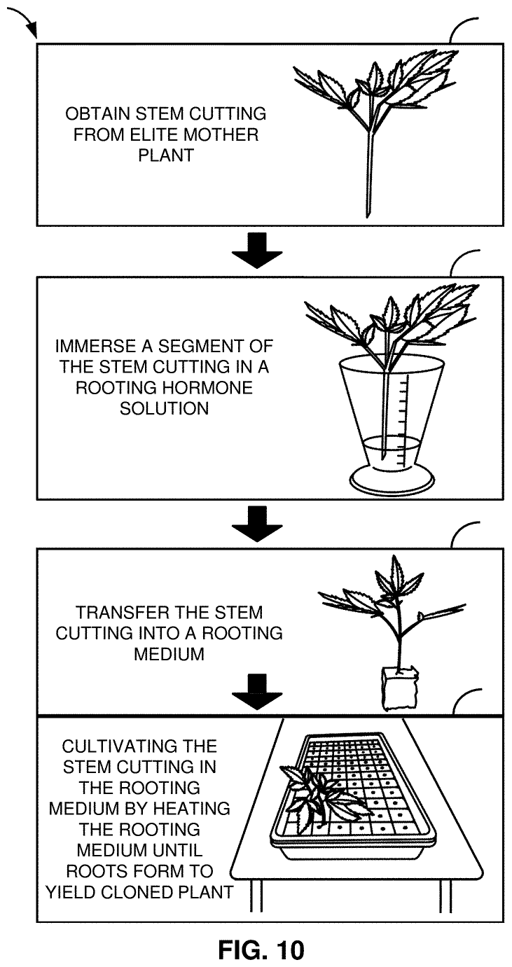

FIG. 10 illustrates certain steps of a method for producing cloned plants of the genus Cannabis from substantially pathogen-free elite mother plants. The elite mother plants can be produced from the method disclosed herein. The steps of method can be considered additional steps of method or a continuation of method.

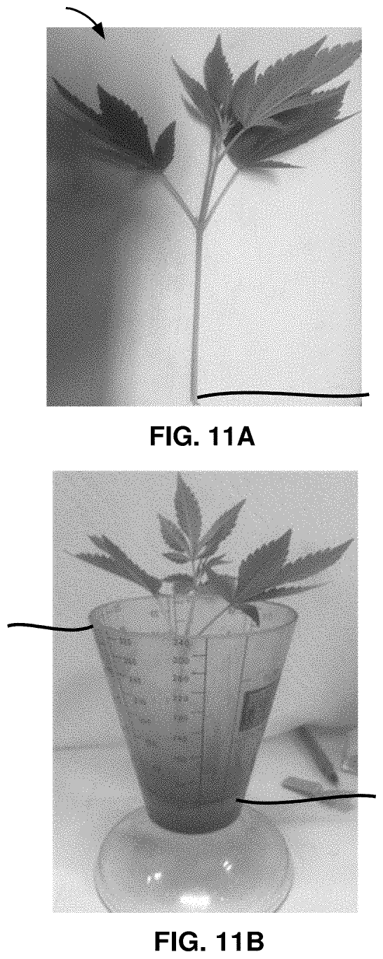

The method can comprise obtaining a stem cutting (see FIGS. 11A and 11B) of the elite mother plant. The stem cutting can be obtained by cutting a main stem or a branch of the elite mother plant using sterilized cutting instruments. In some embodiments, the cutting instruments can comprise pruning shears, scissors, scalpels, or a combination thereof. The stem cutting can measure between approximately 3.0 inches (7.62 cm) and 7.0 inches (17.78 cm) in length.

The method can also comprise immersing at least a segment of the stem cutting in a rooting hormone solution (see FIG. 11B). In one embodiment, the excised or cut end (see FIG. 11A) of the stem cutting can be immersed or dipped in the rooting hormone solution for between approximately 5 seconds and 10 seconds. For example, the rooting hormone solution can be poured into a solution container (e.g., a beaker or cup) and the excised or cut end of the stem cutting can be dipped or immersed in the rooting hormone solution for between approximately 5 seconds and 10 seconds.

In some embodiments, the rooting hormone solution can comprise indole-3-butyric acid (IBA) and 1-napthaleneacetic acid as active ingredients. For example, the rooting hormone solution can be a diluted solution comprising a concentrated rooting hormone solution. The concentrated rooting hormone solution can comprise approximately 1.0% (w/v %) IBA and 0.5% (w/v %) 1-napthaleneacetic acid as active ingredients. The rooting hormone solution can also comprise ethanol and isopropyl alcohol. As a more specific example, the rooting hormone solution can be a diluted solution comprising 10% (v/v %) of Dip 'N Grow® Liquid Rooting Concentrate distributed by Dip 'N Grow Inc. In other embodiments, the rooting hormone solution can be other rooting hormones in the form of powders or gels added to an aqueous solution.

The method can further comprise transferring the stem cutting into a temperature-controlled rooting medium 1 after dipping or immersing the segment of the stem cutting in the rooting hormone solution in step. In one embodiment, the temperature-controlled rooting medium 1 can be a heated rock-wool rooting medium.

The method can also comprise further cultivating the stem cutting in the temperature-controlled rooting medium 1 until roots form to yield a cloned plant in step. In one embodiment, further cultivating the stem cutting in the temperature-controlled rooting medium 1 can comprise heating the rooting medium to a temperature of approximately 80° F. (26.67° C.) using a heating system or heating device and maintaining the temperature of the rooting medium using the heating system or heating device. Moreover, step can also comprise maintaining a relative humidity of 60% using a fog generating machine or fogger and maintaining a relative ambient temperature of approximately 70° F. The stem cutting can be further cultivated in this manner until the stem cutting takes root in approximately 10 days to 16 days. Once the stem cutting has taken root in the rooting medium, the rooted plant can be considered a cloned plant or clone of the elite mother plant.

One unexpected discovery is that cloned plants produced, in part, by a combination of the method and method disclosed herein were, on a population level, healthier than cloned plants produced by other methods. Another unexpected discovery is that cloned plants produced by the methods disclosed herein produced, on average, more cannabinoid or terpenoid-rich plant matter than cloned plants produced by other methods.

FIG. 11A is a black-and-white image of a stem cutting obtained from an elite mother plant. As previously discussed, the stem cutting can be obtained by cutting a main stem or a branch of the elite mother plant using sterilized cutting instruments. In some embodiments, the cutting instruments can comprise pruning shears, scissors, scalpels, or a combination thereof. The stem cutting can measure between approximately 3.0 inches (7.62 cm) and 7.0 inches (17.78 cm) in length. As shown in FIG. 11A, the stem cutting can comprise one or more leaves, nodes, internodes, and branches. Moreover, FIG. 11A shows that the stem cutting can have an excised or cut end.

FIG. 11B is a black-and-white image of a segment of the stem cutting immersed in a rooting hormone solution. For example, the excised or cut end along with a segment of the stem cutting between approximately 0.5 inches (1.27 cm) and 1.0 inches (2.54 cm) in proximity to the cut end can be immersed or dipped into the rooting hormone solution. The segment of the stem cutting can be immersed or dipped in the rooting hormone solution for between approximately 5 seconds and 10 seconds.

As previously discussed, in some embodiments, the rooting hormone solution can comprise indole-3-butyric acid (IBA) and 1-napthaleneacetic acid as active ingredients. For example, the rooting hormone solution 1102 can be a diluted solution comprising a concentrated rooting hormone solution. The concentrated rooting hormone solution can comprise approximately 1.0% (w/v %) IBA and 0.5% (w/v %) 1-napthaleneacetic acid as active ingredients. The rooting hormone solution 1102 can also comprise ethanol and isopropyl alcohol. As a more specific example, the rooting hormone solution can be a diluted solution comprising 10% (v/v %) of Dip 'N Grow® Liquid Rooting Concentrate distributed by Dip 'N Grow Inc. In other embodiments, the rooting hormone solution can be other rooting hormones in the form of powders or gels added to an aqueous solution.

The rooting hormone solution can be poured into a solution container such as a measuring cup or beaker and the segment of the stem cutting in proximity to the excised or cut end can be immersed in the rooting hormone solution for between approximately 5 seconds and 10 seconds. In some embodiments, the solution container can be a polymeric container. In other embodiments, the solution container can be a ceramic or glass container, a stainless steel container, or a combination thereof.

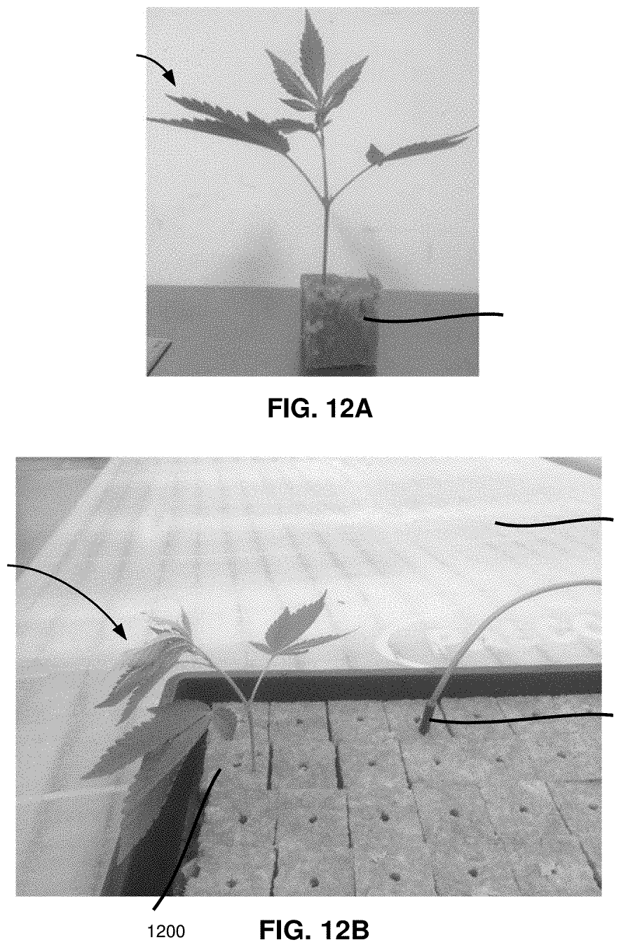

FIG. 12A is a black-and-white image of the stem cutting placed in a rooting medium 1. In one embodiment, the rooting medium 1 can be rock-wool rooting medium. In this and other embodiments, the rooting medium 1 can be or comprise a polymeric rooting medium, soil, pumice, perlite, peat, coir, polymer stabilized rooting plugs, other types of mineral wool, or any combination thereof. The immersed or dipped segment of the stem cutting can be inserted or otherwise placed in the rooting medium 1 soon after being removed from the rooting hormone solution.

FIG. 12B is a black-and-white image showing the stem cutting being further cultivated in a temperature-controlled rooting medium 1 to yield a cloned plant. The rooting medium 1 can be heated to a temperature of approximately 80° F. (26.67° C.) using a heating mat or a hydronic heating system. For example, the heating mat and hydronic heating system can each comprise a heating surface (e.g., a mat surface, heated table top or bench top surface, etc.) and the stem cuttings in rooting medium can be placed on top of the heating surface or in a tray or pan on top of the heating surface (i.e., the rooting medium can be bottom heated). A temperature probe or sensor can also be placed in contact with the rooting medium 1 to gauge or monitor the temperature of the rooting medium. The temperature probe or sensor can be electrically coupled to a programmable controller which can also control a heat-generating apparatus used to heat the heating surface. The programmable controller can turn the heat-generating apparatus on or off in order to heat or cool down the heating surface such that the temperature of the rooting medium 1 (as detected by the temperature probe or sensor 1204) is maintained close to a predetermined set point (e.g., approximately 80° F. or 26.67° C.).

For large scale cloning operations, rooting media 1 comprising stem cuttings can be heated using a hydronic heating system. In one embodiment, the hydronic heating system can be an under-bench heating system distributed by BioTherm, Inc. For small scale cloning operations, rooting media 1 comprising stem cuttings can be heated using an electrical heating mat. In one embodiment, the heating mat can be a Jump Start™ Seedling Heating Mat distributed by Hydrofarm, Inc.

Cultivating the stem cutting can also comprise maintaining a relative humidity of 60% using a fog generating machine or fogger (e.g., a FOGCO™ Revolution Humidification Fan distributed by Fogco Systems, Inc.) and maintaining a relative ambient temperature of approximately 70° F. The stem cutting can be cultivated in this manner until the stem cutting takes root in approximately 10 days to 16 days. Once the stem cutting has taken root in the rooting medium 1, the rooted plant can be considered a cloned plant or clone of the elite mother plant.

### EXAMPLES

### Example 1

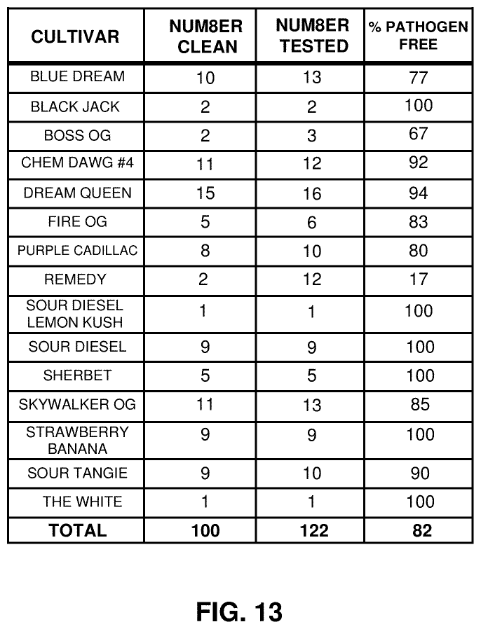

FIG. 13 is a table showing the results of tests conducted in April 2018 on plants of the genus Cannabis produced by the methods described herein using heating for the pretreatment step. As shown in FIG. 13, a total of 122 plants of various cultivars were tested for HpLVd and approximately 82% of the plants tested were pathogen free. Moreover, when testing data for cultivar Remedy is omitted, the percentage increases to approximately 89%. Since previous plant epidemiological studies have estimated the incidence of HpLVd to be as high as approximately 35% (and possibly much higher when infected asymptomatic plants are taken into account) in untreated Cannabis nursery stock plants, a decrease of such incidence to approximately 18% (or 11% when outlier data for cultivars such as Remedy is removed) is a significant advance in the field of Cannabis cultivation. Moreover, the methods described herein can involve testing the plants produced by the process steps disclosed herein (for example, the young plants 802 shown in FIG. 8B) for HpLVd and culling plants that test positive for HpLVd. By doing so, the clones created from the elite mother plants which have survived the culling process will be nearly 100% pathogen free.

As shown in FIG. 13, one unexpected discovery is that the methods disclosed herein are effective for a wide range of Cannabis sativa plants including numerous commercially-valuable cultivars or strains. Moreover, another unexpected discovery is that certain cultivars responded exceptionally well to the methods disclosed herein. For example, plants of the Dream Queen cultivar or strain, the Sour Diesel cultivar or strain, the Sherbet cultivar or strain, and the Strawberry Banana cultivar or strain responded better than other Cannabis sativa cultivars.

### Example 2

Experiments were conducted to test effects of different pretreatments disclosed herein on plants to eliminate HpLVd. The source material for these experiments originated from tissue cultured mericlone lines known to be infected with HpLVd from prior testing. 16 different strains were used in these experiments, most commonly SPK, BD, SKY, and RDY which together accounted for 70% of all meristems excised. All plant material sourced from whole plants (ex-vitro) was created by rooting and acclimatizing HpLVd-infected tissue culture plants and growing them out to a suitable size before treatment. All treatments using in-vitro tissue cultured plants were grown in glass test tubes on full strength MS medium with 30 g/L sugar solidified with 8 g/L phytogel.

For all treatments, apical meristems less than 0.5 mm in diameter were excised from the plant tissue at the end of the treatment period. The post-treatment excised meristems were cultured in test tubes and grown under 18-hour light on full strength MS medium with 30 g/L sugar solidified with 8 g/L phytogel until large enough to test.

Testing for HpLVd was done via RNA extraction and Reverse Transcription Polymerase Chain Reaction (RT-PCR). One round of testing was conducted in February 2019, with two more rounds in August and September 2019.

A total of 428 meristems were excised, of which survived, for an overall survival rate of 41.5% (Table 1). About two thirds (128) of the surviving meristems have been tested for HpLVd. The remaining untested plants were too small to test for HpLVd at the end of the study.

TABLE 1
Summary of results aggregated by treatment type.

| Treat- | Number | Number | Number | Number | Survival | % HpLVd- |
|---|---|---|---|---|---|---|
| ment | Excised | Surviving | Tested | Infected | % | Free |
| Heat | 148 | 48 | 36 | 10 | 32.4 | 72.2 |
| Cold | 190 | 77 | 33 | 11 | 40.5 | 66.7 |
| Electricity | 74 | 32 | 23 | 2 | 41.2 | 91.3 |
| Overall | 428 | | 128 | 35 | 41.5 | 72.6 |

### Example 3

Experiments were performed using heat treatment as a pretreatment in the protocol of Example 2. The Heat Treatment was carried out on whole plants (ex-vitro) in a growth chamber kept at 100 degrees Fahrenheit 24 hours a day under 24-hour light. At 2, 3 and 4 weeks of heat treatment nodes were harvested from the same donor plants, sterilized and excised on the same day treatment was finished (Table 2). The three and four-week heat treatments increased the percentage of HpLVd-free plants obtained post-treatment.

TABLE 2
Effectiveness of heat treatments of different lengths
(2-4 weeks) on HpLVd eradication.

| Duration | | | | | | % |
|---|---|---|---|---|---|---|
| of Heat | Number | Number | Number | Number | Survival | HpLVd- |
| Treatment | Excised | Surviving | Tested | Infected | % | Free |
| 2 Weeks | 71 | 24 | 18 | 7 | 33.8 | 61.1 |
| 3 Weeks | 46 | 9 | 6 | 0 | 19.6 | 100 |
| 4 Weeks | 31 | 15 | 12 | 3 | 48.4 | 75 |

### Example 4

Experiments were performed using cold treatment as a pretreatment in the protocol of Example 2. The Cold Treatment was performed by placing sterile tissue culture plants grown in test tubes in a fridge lit for 18 hours per day. The temperature inside the fridge was kept at 40 degrees F.+/−5 degrees. Many of the cultures grew significantly in size and formed roots during treatment.

None of the 8 meristems tested from the 9-month and 1-year cold treatment periods tested positive for HpLVd.

TABLE 3
Effectiveness of cold treatments on HpLVd eradication.

| Duration | | | | | | % |
|---|---|---|---|---|---|---|
| of Cold | Number | Number | Number | Number | Survival | HpLVd- |
| Treatment | Excised | Surviving | Tested | Infected | % | Free |
| 1 Month | 46 | 16 | 16 | 7 | 34.8 | 56.3 |
| 3 Months | 38 | 18 | 9 | 4 | 47.4 | 55.6 |
| 6 Months | 33 | 10 | 0 | No Data | 30.3 | No Data |
| 9 Months | 33 | 12 | 3 | 0 | 36.4 | 100 |
| 1 Year | 40 | 21 | 5 | 0 | 52.5 | 100 |

### Example 5

Experiments are performed using electricity as a pretreatment in the protocol of Example 2. The Electricity Treatments are applied to ex-vitro plants in a gel electrophoresis tank. The plant material is kept completely submerged in a 1M Sodium Chloride solution to carry the electrical current for 20 minutes at currents of 50, 75 and 100 milliamperes (mA). Current is supplied from a DC electrophoresis power supply capable of delivering precise levels of electrical current.

### Example 6

Experiments are done using the anti-viral compound Ribavirin to eradicate viruses and viroids. Ribavirin was used in levels of 20 and 80 mg/L sterile-filtered into the culture medium after autoclaving.

The impact of adding the anti-viral drug Ribavirin was mainly to reduce the survival rate of the plants (Table 4). There was also a decrease in HpLVD-free plants with increasing Ribavirin concentration. An associated no-treatment control group performed better both in survival and percentage of HpLVd-free plants.

TABLE 4
Summary of results of treatments using Ribavirin. Note the
negative impact on both meristem survival and eradication
of HpLVd with increasing Ribavirin concentration.

| | | | | | | % |
|---|---|---|---|---|---|---|
| Ribavirin | Number | Number | Number | Number | Survival | HpLVd- |
| (mg/L) | Excised | Surviving | Tested | Infected | % | Free |
| 0 | 49 | 34 | 21 | 2 | 69.4 | 90.5 |
| 20 | 56 | 34 | 20 | 4 | 60.7 | 80.0 |
| 80 | 51 | 6 | 3 | 2 | 11.7 | 33.3 |

### Example 7

Experiments are done using heat treatment combined with electrotherapy as a pretreatment using the protocol of Example 2. The plant is pretreated with heat treatment and then immediately treated with electricity.

### Example 8

Experiments are done using cold treatment combined with electrotherapy as a pretreatment using the protocol of Example 2. The plant is pretreated with electrotherapy, followed by meristem excision. Once the meristem has grown out, the plant is pretreated with cold treatment.

### Example 9

Experiments are done using both heat and cold treatments as a pretreatment using the protocol of Example 2. The plant is treated with heat and then undergoes meristem excision. Then plant is treated with cold and then undergoes another meristem excision.

### Example 10

Experiments are done using heat, cold and electrotherapy treatments as a pretreatment step using the protocol of Example 2. The plants are treated with heat, followed by electrotherapy, followed by meristem excision, and then once the plants have grown out, the plants are put through cold treatment and another meristem excision.

### Example 11

Experiments are done using heat treatment and anti-viral treatment in the protocol of Example 2. 10-75 mg/L of the anti-viral compound Ribavirin is added to the culture medium used to regenerate the excised meristem after heat treatment.

### Example 12

Experiments are done using heat treatment, anti-viral treatment, and electrotherapy in the protocol of Example 2. 10-75 mg/L of the anti-viral compound Ribavirin is added to the culture medium used to regenerate the excised meristem after heat treatment. The plants are then subject to electrotherapy treatment.

### Example 13

Experiments are done using cold treatment and anti-viral treatment in the protocol of Example 2. The plants are treated with cold while in culture medium containing 25-75 mg/L Ribavirin.

### Example 14

Experiments are done using cold treatment, anti-viral treatment, and electrotherapy treatment as a pretreatment step in the protocol of Example 2. The plants are treated with cold while in culture medium containing 25-75 mg/L Ribavirin. Then the plants are treated with electrotherapy.

### Example 15

Experiments are done using electrotherapy and anti-viral compound treatment. 10-75 mg/L of anti-viral compound is added to the culture medium used to regenerate the excised meristem after electrotherapy treatment.

### Example 16

Experiments are done using heat treatment, cold treatment, electrotherapy, and an anti-viral compound as a pretreatment step in the protocol of Example 2. Heat treatment is followed by electrotherapy, followed by meristem excision and a cultured in media containing 25-75 mg/L Ribavirin. Once the plants have grown out they are then placed in cold treatment in media with or without Ribavirin. Once the treatment is completed the meristems are again excised and cultured on media with or without Ribavirin.

A number of embodiments have been described. Nevertheless, it will be understood by one of ordinary skill in the art that various modifications may be made without departing from the spirit and scope of the embodiments. In addition, the flowcharts or logic flows depicted in the figures do not require the particular order shown, or sequential order, to achieve desirable results. In addition, other steps or operations may be provided, or steps or operations may be eliminated, from the described flows, and other components may be added to, or removed from, the described systems. Accordingly, other embodiments are within the scope of the following claims.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure. Accordingly, the specification and/or drawings may be regarded in an illustrative rather than a restrictive sense.

### REFERENCES

Barbara, D. J., and A. N. Adams. “Hop latent viroid.” Viroids. A. Hadidi, R. Flores, J W Randles, and J S Semancik, eds. CSIRO Publ., Collingwood, Australia (2003): 213-217.
Diener, Theodor 0. “Origin and evolution of viroids and viroid-like satellite RNAs.” Virus genes 11.2-3 (1995): 119-131.
Gamborg, 0. L., et al. “Plant tissue culture media.” In Vitro Cellular & Developmental Biology-Plant 12.7 (1976): 473-478
Matoušek, J, et al. “The variability of hop latent viroid as induced upon heat treatment.” Virology 287.2 (2001): 349-358.
Matoušek, J. “Hop latent viroid (HLVd) microevolution: an experimental transmission of HLVd “thermomutants” to solanaceous species.” Biologia plantarum 46.4 (2003): 607-610.
Pethybridge, Sarah J., et al. “Viruses and viroids infecting hop: Significance, epidemiology, and management.” Plant disease 92.3 (2008): 324-338.
Puchta, Holger, Karla Ramm, and Heinz L. Sanger. “The molecular structure of hop latent viroid (HLV), a new viroid occurring worldwide in hops.” Nucleic acids research 16.10 (1988): 4197-4216.
White, Philip R. A handbook of plant tissue culture. Vol. 56. No. 2. LWW (1943).

What is claimed is:

1. A method of producing plants of the genus Cannabis, the method comprising:
pretreating a progenitor plant of the genus Cannabis, resulting in a pretreated plant;
surface sterilizing a shoot segment of the pretreated plant with a bleach solution;
excising a meristematic tip of the shoot segment; and
transferring the meristematic tip into a culturing plate comprising at least one plant hormone capable of inducing formation of a whole plant from the meristematic tip.

2. The method of claim 1, wherein the pretreating step includes one or more of heating the progenitor plant within a heating chamber, cooling the progenitor plant within a cooling chamber, delivering an electrical current to the progenitor plant, and/or applying an anti-viral compound to the progenitor plant.

3. The method of claim 1, wherein the medium comprises a supplemented Murashige and Skoog culture medium and wherein the at least one plant hormone comprises one or more of 1.0 mg/L of benzyladenine, 0.1 mg/L of naphthaleneacetic acid, and 0.1 mg/L of gibberellic acid.

4. The method of claim 1, wherein excising the meristematic tip of the shoot segment comprises excising an apical portion of the shoot segment equal to or less than approximately 0.5 mm in size, wherein the apical portion of the shoot segment comprises meristem tissue.

5. The method of claim 1, wherein a height dimension of the progenitor plant is between 6 inches and 18 inches as measured from a soil surface.

6. The method of claim 1, wherein surface sterilizing the shoot segment of the pre-treated plant comprises immersing the shoot segment in the bleach solution for between approximately 10 minutes and 20 minutes.

7. The method of claim 6, wherein the bleach solution comprises approximately 2.475% (w/v %) of sodium hypochlorite.

8. The method of claim 1, further comprising transferring a plantlet grown from the meristematic tip from the culturing plate into a test tube comprising additional supplemented Murashige and Skoog culture medium after 21 days to 30 days.

9. The method of claim 8, further comprising transferring the plantlet growing in the test tube into a large-tissue culture vessel comprising Murashige and Skoog culture medium after 28 days to 56 days.

10. The method of claim 9, further comprising:
transferring the plantlet growing in the large-tissue culture vessel into a first rooting medium after 28 days to 56 days to yield a young elite mother plant;
transferring the young elite mother plant and at least a portion of the first rooting medium into a second rooting medium after 10 days to 16 days; and
growing the young elite mother plant in the second rooting medium between 7 days and 28 days to yield an elite mother plant.

11. A pathogen-free, surface-sterile, regenerated plant of the genus Cannabis produced by a process comprising the steps of:
pretreating a progenitor plant of the genus Cannabis resulting in a pretreated plant;
surface sterilizing a shoot segment of the pretreated plant with a bleach solution;
excising a meristematic tip of the shoot segment; and
transferring the meristematic tip into a culturing plate comprising a supplemented Murashige and Skoog culture medium for further culturing, wherein the supplemented Murashige and Skoog culture medium comprises benzyladenine, naphthaleneacetic acid, and gibberellic acid.

12. The plant of claim 11, wherein the pretreating step includes one or more of heating the progenitor plant within a heating chamber, cooling the progenitor plant within a cooling chamber, delivering an electrical current to the progenitor plant, and/or applying an anti-viral compound to the progenitor plant.

13. The plant of claim 11, wherein the supplemented Murashige and Skoog culture medium comprises 1.0 mg/L of benzyladenine, 0.1 mg/L of naphthaleneacetic acid, and 0.1 mg/L of gibberellic acid.

14. The plant of claim 11, wherein the process of producing the plant further comprises excising the meristematic tip of the shoot segment by excising an apical portion of the shoot segment equal to or less than approximately 0.5 mm in size, wherein the apical portion of the shoot segment comprises meristem tissue.

15. The plant of claim 11, wherein a height dimension of the progenitor plant is between 6 inches and 18 inches as measured from a soil surface.

16. The plant of claim 11, wherein the process of producing the plant further comprises surface sterilizing the shoot segment of the pretreated plant by immersing the shoot segment in the bleach solution for between 10 minutes and 20 minutes.

17. The plant of claim 16, wherein the bleach solution comprises approximately 2.475% (w/v %) of sodium hypochlorite.

18. The plant of claim 11, wherein the process of producing the plant further comprises transferring a plantlet grown from the meristematic tip from the culturing plate into a test tube comprising additional supplemented Murashige and Skoog culture medium after 21 days to 30 days.

19. The plant of claim 18, wherein the process of producing the plant further comprises transferring the plantlet growing in the test tube into a large-tissue culture vessel comprising Murashige and Skoog culture medium after 28 days to 56 days.

20. The plant of claim 19, wherein the process of producing the plant further comprises:
transferring the plantlet growing in the large-tissue culture vessel into a first rooting medium after 28 days to 56 days to yield a young elite mother plant;
transferring the young elite mother plant and at least a portion of the first rooting medium into a second rooting medium after 10 days to 16 days; and
growing the young elite mother plant in the second rooting medium between 7 days and 28 days to yield an elite mother plant.

21. A cloned plant of the genus Cannabis produced by a process comprising the steps of:
pretreating a progenitor plant of the genus Cannabis, resulting in a pretreated plant;
surface sterilizing a shoot segment of the pretreated progenitor plant with a bleach solution;
excising a meristematic tip of the shoot segment;
transferring the meristematic tip into a culturing plate comprising a supplemented Murashige and Skoog culture medium, wherein the supplemented Murashige and Skoog culture medium comprises benzyladenine, naphthaleneacetic acid, and gibberellic acid;
transferring a plantlet grown from the meristematic tip from the culturing plate into a test tube comprising additional supplemented Murashige and Skoog culture medium after 21 days to 30 days;
transferring the plantlet growing in the test tube into a large-tissue culture vessel comprising Murashige and Skoog culture medium after 28 days to 56 days;
transferring the plantlet growing in the large-tissue culture vessel into a first rooting medium after 28 days to 56 days to yield a young elite mother plant
transferring the young elite mother plant and at least a portion of the first rooting medium into a second rooting medium after 10 days to 16 days;
growing the young elite mother plant in the second rooting medium between 7 days and 28 days to yield an elite mother plant;
obtaining a stem cutting of the elite mother plant;
immersing at least a segment of the stem cutting in a rooting hormone solution; and
transferring the stem cutting into a temperature-controlled rooting medium and further cultivating the stem cutting in the temperature-controlled rooting medium until roots form to yield the cloned plant.

22. The cloned plant of claim 21, wherein the pretreating step includes one or more of heating the progenitor plant within a heating chamber, cooling the progenitor plant within a cooling chamber, delivering an electrical current to the progenitor plant, and/or applying an anti-viral compound to the progenitor plant.

23. The cloned plant of claim 21, wherein the supplemented Murashige and Skoog culture medium comprises 1.0 mg/L of benzyladenine, 0.1 mg/L of naphthaleneacetic acid, and 0.1 mg/L of gibberellic acid.

24. The cloned plant of claim 21, wherein the process of producing the cloned plant further comprises excising the meristematic tip of the shoot segment by excising an apical portion of the shoot segment equal to or less than approximately 0.5 mm in size, wherein the apical portion of the shoot segment comprises meristem tissue.

25. The cloned plant of claim 21, wherein a height dimension of the progenitor plant is between 6 inches and 18 inches as measured from a soil surface.

26. The cloned plant of claim 21, wherein the process of producing the cloned plant further comprises surface sterilizing the shoot segment of the pretreated plant by immersing the shoot segment in the bleach solution for between 10 minutes and 20 minutes.

27. The cloned plant of claim 26, wherein the bleach solution comprises approximately 2.475% (w/v %) of sodium hypochlorite.

28. The cloned plant of claim 21, wherein the process of producing the cloned plant further comprises immersing the segment of the stem cutting in the rooting hormone solution for between 5 seconds and 10 seconds.

29. The cloned plant of claim 21, wherein the rooting hormone solution comprises indole-3-butyric acid and 1-napthaleneacetic acid as active ingredients.
